(19) United States
(12) Patent Application Publication
(10) Pub. No.: US 2005/0245911 A1
(43) Pub. Date: Nov. 3, 2005

(54) User selectable grip for use with ophthalmic surgical handpieces

(76) Inventors: David Walter Wright, Littleton, CO (US); Lee William Travis, Littleton, CO (US)

Correspondence Address: Bausch & Lomb Incorporated, One Bausch & Lomb Place, Rochester, NY, 14604-2701, US

(21) Appl. No.: 10/835,039

(22) Filed: Apr. 29, 2004

(51) Int. Cl. A61B 17/00

(52) U.S. Cl. 606/1

(57) ABSTRACT

A user selectable grip 10 for use with an ophthalmic surgical handpiece 12 includes an elongated grip 20 with an outer surface to be grasped by user. The grip 20 has a void extending the length of the grip 20, such that the void matingly fits over a portion 14 of a surgical handpiece 12, such that the grip 20 is removable from surgical handpiece 12.

### BACKGROUND OF THE INVENTION

1. Field of the Invention

The present invention is related to ophthalmic surgical handpieces and more specifically, to ophthalmic surgical handpieces having user removable grips.

2. Description of Related Art

Presently, ophthalmic surgical handpieces have grips, which are permanently and integrally incorporated into the handpiece. The shape of the grip is pre-determined by the manufacturer and the user must accommodate the shape of the handpiece and its accompanying grip for use in surgery.

These grips also take the form of a hard surface, such as titanium or other metal or plastic and have such surface textures as knurled surfaces.

Given the delicate nature of ophthalmic surgery and the varying preferences of different surgeons, it would be advantageous to provide the surgeon with a user selectable grip having a geometric outer shape with which the surgeon is comfortable and which allows the handpiece to be efficiently manipulated to provide for a fast and safe surgery.

### BRIEF DESCRIPTION OF DRAWINGS

FIG. 1 is an exploded partial perspective view of a grip in accordance with the present invention separated from a surgical handpiece;

FIG. 2 is a perspective view of a grip in accordance with the present invention fitted on a surgical handpiece;

FIG. 3 is a cross-sectional view of FIG. 2 taken along line 3-3 of FIG. 2;

FIG. 4 are perspective views of several alternative grip shapes for grips in accordance with the present invention; and

FIG. 5 is a perspective view of an embodiment of a grip in accordance with the present invention.

### DETAILED DESCRIPTION OF THE PREFERRED EMBODIMENT

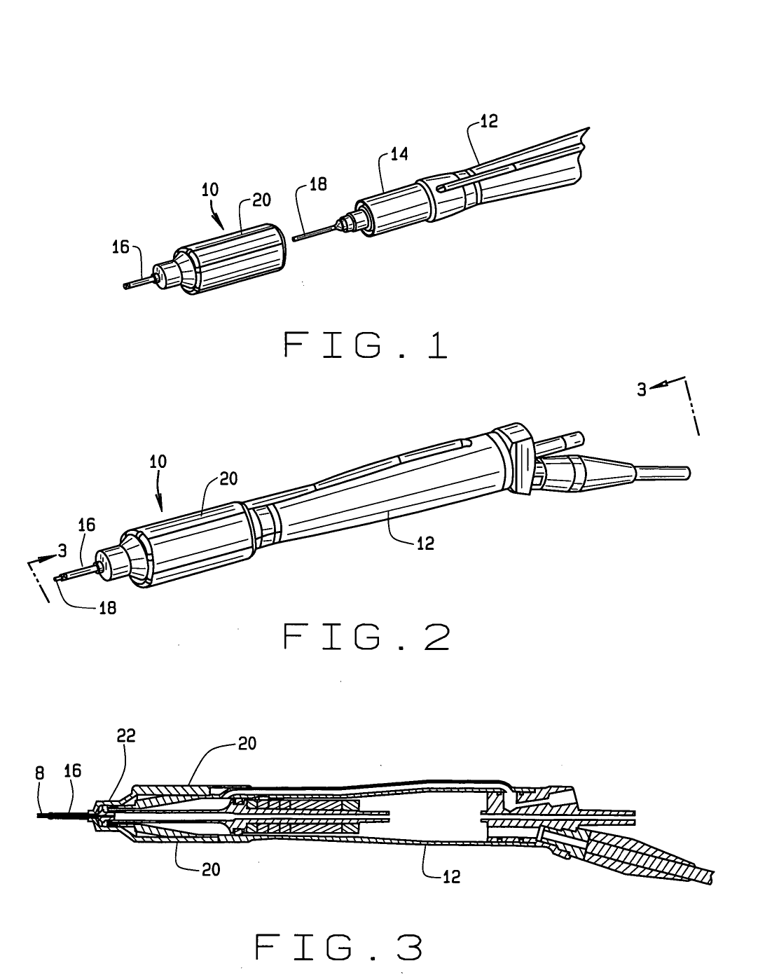

FIG. 1 shows a user selectable grip 10 for use with an ophthalmic surgical handpiece 12. Grip 10 is shown in exploded relationship with handpiece 12 where grip 10 has a void that cannot be seen extending within the grip 10. Grip 10 matingly fits over portion 14 of the phacoemulsification handpiece 12 and a sleeve 16 of grip 10 fits over a needle 18. The irrigation sleeve 16 is attached to the elongated grip portion 20 that has an outer surface to be grasped by a user. Preferably, the grip 20 and sleeve 16 are removable from the handpiece by the user, such as by simply pulling on grip 20. It is to be understood that grip 20 may be attached to handpiece 12 by other than the shown press fit. Other means of attachment include threaded attachment or some form of detent or retention ring attachment. One advantage of a threaded connection would be the capability of precise placement of the sleeve relative to the needle 18.

FIG. 2 shows a perspective view of a phacoemulsification handpiece 12 with grip 10 attached to the handpiece 12. Such as shown in more detail below, with respect to an alternative embodiment of the present invention, grip 20 may have many different outer geometric shapes which may be selected by the user depending on the user's preference and the operation to be performed. Preferably grip 20 and sleeve 16 are formed or molded of the same material, which material may be silicone, urethane, polyvinylchloride, Delrin, or other suitable materials. Preferably, the grip 20 slidingly attaches to the handpiece 12 via press fit, as shown clearly in FIG. 3. Grip 10 preferably includes a hub section 22 between sleeve 16 and elongated grip 20.

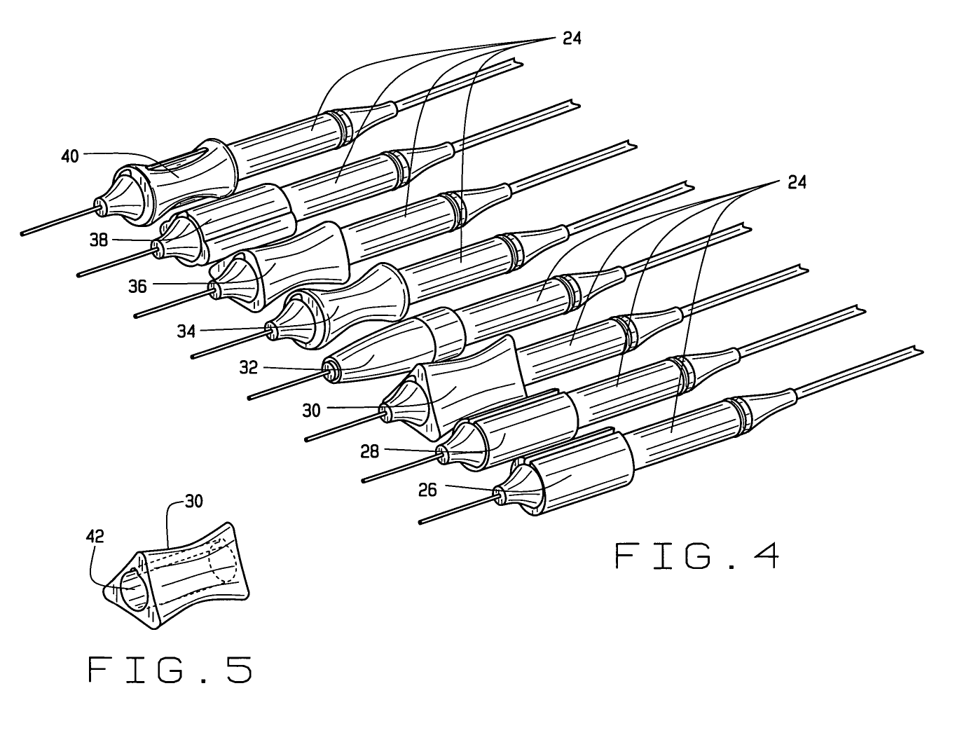

FIG. 4 shows an array of different potential grip geometries fitted to a vitrectomy cutter 24. Elongated grips 26, 28, 30, 32, 34, 36, 38, and 40 each have an outer surface to be grasped by a user. The grips 26-40 have voids extending the length of the grip such that the void matingly fits over a portion of the surgical handpiece 24 such that the grip is removable from the surgical handpiece. As can be seen from the grips shown in FIG. 4, a wide variety of different outer grip geometric shapes are available for selection by a surgeon. The grip to be used may be selected by user preference and by the type of manipulation needed to be made to the handpiece in surgery. While the grips are shown attached to a vitrectomy cutter in FIG. 4 and a phacoemulsification handpiece in FIGS. 1-3, it is to be understood that the grips may be made to fit any number of different surgical handpieces, such as laser probes, pneumatic scissors, irrigation devices, or other such known surgical handpieces. Another advantage of the inventive grips of the present invention, is that the grips are disposable and therefore the surgeon may have a new, unworn grip for use in each surgery.

FIG. 5 shows a grip 30 detached from its mating handpiece and showing a void 42, which extends the length of the grip 30 such that the void 42 matingly fits over a portion of the surgical handpiece 24 such that the grip 30 is removable from the surgical handpiece 24.

1. A user selectable grip for use with ophthalmic surgical handpieces comprising:
an elongated grip with an outer surface to be grasped by a user; and
wherein the grip has a void extending the length of the grip, such that the void matingly fits over a portion of a surgical handpiece, such that the grip is removable from the surgical handpiece.

2. The grip of claim 1, wherein the grip is formed of a material taken from the group consisting of silicone, urethane, polyvinylchloride, and Delrin.

3. The grip of claim 1, wherein the grip slidingly attaches to the handpiece via a press fit.

4. The grip of claim 1, wherein the grip threads onto the handpiece.

5. The grip of claim 1, wherein the grip is disposable.

6. A user selectable grip for use with a phacoemulsification handpiece comprising:
an elongated grip with an outer surface to be grasped by a user;
an irrigation sleeve attached to the grip for surrounding a needle;
wherein the grip has a void extending within the grip, such that the grip matingly fits over a portion of a phacoemulsification handpiece and the sleeve fits over a needle; and
wherein the grip and sleeve are removable from the handpiece.

7. The grip of claim 6, wherein the grip is formed of a material taken from the group consisting of silicone, urethane, polyvinylchloride, and Delrin.

8. The grip of claim 6, wherein the grip slidingly attaches to the handpiece via a press fit.

9. The grip of claim 6, wherein the grip threads onto the handpiece.

10. The grip of claim 6, wherein the grip is disposable.
